(19) United States
(12) Patent Application Publication
(10) Pub. No.: US 2018/0286504 A1
(43) Pub. Date: Oct. 4, 2018

(54) CHALLENGE VALUE ICONS FOR RADIOLOGY REPORT SELECTION

(71) Applicant: KONINKLIJKE PHILIPS N.V., EINDHOVEN (NL)

(72) Inventor: KAREN IRENE TROVATO, PUTNAM VALLEY, NY (US)

(21) Appl. No.: 15/762,113

(22) PCT Filed: Sep. 16, 2016
(86) PCT No.: PCT/IB2016/055520

§ 371 Date: Mar. 22, 2018

Related U.S. Application Data
(60) Provisional application No. 62/233,460, filed on Sep. 28, 2015.

(51) Int. Cl. G16H 15/00; G06F 3/0482; G16H 40/40; G16H 30/20

(52) U.S. Cl. CPC G16H 15/00; G06F 3/0482; G06F 17/28; G16H 30/20; G16H 40/40

(57) ABSTRACT

A radiology workstation (14) includes a processor (16), user input devices (24, 26, 28), and at least one display device (20, 22) that displays a work list (32) of radiology examination reading tasks. A radiology examination reading task is selected from the work list, and radiology images are retrieved from a Picture Archiving and Communication System (PACS) and displayed. Entry of a radiology report is received via the at least one user input device. A challenge level assessment component (60) generates prospective challenge levels (88) for radiology examination reading tasks prior to entry of the radiology reports for the reading tasks, and the radiology workstation displays the work list with indicators (50, 54) of the prospective challenge levels generated by the challenge level assessment component for the radiology examination reading tasks. The indicators may be, for example, color indicators, icon indicators, colored icon indicators, or textual indicators.

### FIELD

The following relates generally to the radiology arts, radiology reading arts, medical picture archiving and communications system (PACS) arts, radiology workstation arts, radiology workstation user interfacing arts, and related arts.

### BACKGROUND

Radiologists are highly specialized medical professionals, and as such are expected to maintain a high throughput. In a typical work environment, the radiologist is seated at a PACS workstation running radiology workstation software such as the Philips iSite PACS workstation system (available from Koninklijke Philips N.V., Eindhoven, the Netherlands). A work list is maintained listing the radiology reading tasks to be performed by the radiologist (or team of radiologists) for that work shift. The work list provides limited information (e.g. patient name, gender, date of birth). When a task is selected, the workstation provides further, but still limited, information available to the PACS, such as the reason for examination and any other available patient data, and enables the radiologist to view the acquired images at high resolution. The radiologist is liable for reviewing every image, and in many cases, a three-dimensional (i.e. volumetric) image set is analyzed slice-by-slice, including every slice, rather than on the basis of a 3D rendering. The radiologist dictates a report of findings (describing facts related to visualized anatomy and pathology within the radiology report) which is sent to the patient's physician and is also stored on the PACS.

To maintain an efficient work flow, and due to the highly specialized nature of the PACS database, the amount of patient information available to the radiologist is limited. The radiology workstation often is not connected with the more general-purpose Electronic Medical Record (EMR) or Electronic Health Record (EHR), and so the radiologist does not have ready access to patient information that is not directly related to past radiological examinations. The patient data available to the radiologist is typically limited to patient demographic data (age/date of birth, gender, ethnicity), reason for the radiological examination, the radiological images to be read, and any associated image metadata. Prior radiology reports for the patient (if any) are also available on the PACS workstation, and may be referenced by the radiologist if relevant to the reading (e.g. assessing growth or shrinkage of a tumor since the last imaging session).

While in principle the radiologist may be authorized to access additional patient information on other systems (e.g. the EMR or EHR), in practice time constraints usually limit the radiologist to working only on data available within the PACS. This may include images obtained at the current site, but not those from other sites. To provide context, a typical radiology department may expect the radiologist to perform a complete x-ray or ultrasound reading, including reviewing every image, making medical determinations, and dictating and filing the radiology report, in a time frame of about 1.5-2.0 minutes. A more complex reading, such as a multi-slice computed tomography (CT) or magnetic resonance imaging (MRI) reading, may be expected to be performed in about 5-7 minutes. These are merely illustrative expected reading times and longer or shorter expected reading times may be instituted for a given radiology department. A given radiology reading task also may take longer (or shorter) than these expected times but on average, the radiologist is expected to meet these time frames to be operating at an acceptable level of efficiency and in order to complete the radiology reading task list for a given shift.

In a typical evaluation paradigm, each radiology reading task has a compensation value designated by Relative Value Units (RVUs). For example, in some institutions, a CT reading is assigned 4 RVU points, an MRI reading is assigned 8 RVU points, and an x-ray reading is assigned 1 RVU point. The radiologist is expected to perform readings with a certain number of total RVU points per shift. While some institutions provide finer-grained RVU assessment, in many institutions the RVU points are assigned based on modality (e.g. CT, MRI, X-ray) alone.

### BRIEF SUMMARY

In accordance with one illustrative example, a radiology workstation includes a workstation electronic processor, at least one user input device, and at least one display device. The radiology workstation is configured to: display a work list of radiology examination reading tasks; receive via the at least one user input device a selection of a radiology examination reading task from the work list; retrieve one or more radiology images of the selected radiology examination reading task from a Picture Archiving and Communication System (PACS); and display the retrieved radiology images. A challenge level assessment component comprises an electronic processor programmed to generate prospective challenge levels for the radiology examination reading tasks of the work list. The radiology workstation is configured to display the work list of radiology examination reading tasks with indicators of the prospective challenge levels generated by the challenge level assessment component for the radiology examination reading tasks. The indicators may be, for example, color indicators, icon indicators, colored icon indicators, or textual indicators.

In accordance with another illustrative example, a radiology reading method comprises: generating, using an electronic processor, a prospective challenge level for each radiology examination reading task of a work list of radiology examination reading tasks that have not yet been performed; and displaying the work list on a radiology workstation with each radiology examination reading task of the displayed work list annotated by an indicator of the prospective challenge level generated for the radiology examination reading task. The radiology reading method may further comprise: receiving from the displayed work list a selection of a radiology examination reading task via the radiology workstation; retrieving from a Picture Archiving and Communications System (PACS) one or more radiology images of the selected radiology examination reading task; displaying the retrieved one or more radiology images on the radiology workstation; receiving via the radiology workstation a radiology report for the selected radiology examination reading task; storing the radiology report for the selected radiology examination reading task on the PACS whereby the selected radiology examination reading task is converted to a completed radiology examination reading task; and after the storing, updating the work list by removing the completed radiology examination reading task from the work list and displaying the updated work list on the radiology workstation.

One advantage resides in providing a radiology workstation with a more efficient user interface.

Another advantage resides in providing a radiology workstation providing for more efficient allocation of radiology examination reading tasks to one or more radiologists.

Further advantages of the present invention will be appreciated to those of ordinary skill in the art upon reading and understand the following detailed description. It will be appreciated that a given embodiment may provide none, one, two, or more of these advantages.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a radiology workstation including a prospective challenge level assessment component as disclosed herein. Naturally, this could be performed as a client-server system, where the user interface is remote from the physical computer.

FIGS. 2 and 3 diagrammatically illustrate a work list displayed by the radiology workstation of FIG. 1 with challenge level indicators comprising colorized or shaded challenge level icons (FIG. 2) or challenge level background shading or colors (FIG. 3) indicating challenge levels prospectively estimated by the prospective challenge level assessment component for radiology examination reading tasks of the work task.

FIG. 4 diagrammatically illustrates a suitable mapping from patient age to challenge value level (or to challenge level value component).

FIG. 5 diagrammatically illustrates a method for updating radiologist-specific weights of the prospective challenge level assessment component of FIG. 1.

### DETAILED DESCRIPTION

Some radiology departments employ a “first in, first out” workflow, in which radiology reading tasks are performed in the order they arrive. However, this approach can overstress a radiologist if, for example, the radiologist is forced to perform several complex and mentally taxing readings in a row.

To reduce the stress level, many radiology departments permit the radiologist to choose the next reading to perform from a work list of the radiology examinations. This allows the radiologist to interleave difficult and easier reading tasks in order to reduce stress. Reduced radiologist stress is expected to lead to more accurate readings, and ultimately to higher efficiency. Radiologists typically read between 3200 to over 6000 ‘RVU points’ per year, where “RVU” denotes “Relative Value Units”. Higher numbers generate higher revenue, and radiologists are encouraged to produce a high number of RVU points per shift. Some radiologists, given such freedom, tend to follow a particular pattern, such as performing two difficult reading tasks followed by two easy reading tasks. The radiologist who has just completed a difficult reading task can elect to perform an easier (i.e. less mentally taxing) reading task next. On the other hand, after performing a few easier reading tasks the radiologist may be reinvigorated to perform a complex, mentally taxing reading task next. A given radiologist may also preferentially choose certain tasks which the radiologist finds to be easier due to individualized expertise.

In many medical institutions, RVU points are assigned for each medical procedure code as these codes are used for billing. Two common medical procedure coding systems are Current Procedural Terminology (CPT) codes and Healthcare Common Procedure Coding System (HCPCS) codes. In the case of medical imaging procedures, the codes are delineated by imaging modality, anatomical region, and perhaps other features such as clinical task. Each radiological reading task falls under a certain medical procedure code which has an assigned RVU point value. The RVU point value for a given reading task identified by procedure code is intended to be a compensation metric. Ideally, the RVU points should be proportional to the expected difficulty of the reading task, which roughly translates to an expected reading time (intuitively, a more difficult task should take longer and be more highly compensated).

In practice however, other factors impact the RVU points assignments, such that the RVU points value is only a rough metric for reading task difficulty. One factor is that a single procedure code may cover a range of radiological examinations of varying difficulty. The difficulty of a given reading task may also vary widely depending upon patient particulars (e.g. patient age, physical condition, chronic conditions). Yet another factor that may impact the RVU point assignment is imaging system infrastructure cost. Various market forces may also impact RVU points. For example, a medical institution providing an imaging examination not available elsewhere in the geographical region may charge more for that unique procedure, so that those reading tasks are assigned higher RVU points; whereas, a widely available procedure such as a mammogram may be assigned a lower RVU points value even though the mammogram reading task may be fairly difficult.

When working a shift, the radiologist will generally be aware of the RVU points allocated for each type of reading task on the work list based on its corresponding medical procedure code. The radiologist may also be at least qualitatively aware of other factors that may impact the actual difficulty of a reading task (i.e. the time to perform the reading task). The radiologist may be unaware of other factors that impact the actual difficulty of a reading task, which are not listed in the radiology reading task list. Based on the RVU points and these other limited sources of information, the radiologist selects a next reading task to perform. This approach has the disadvantages that the radiologist is unable to accurately assess task difficulty, and moreover the radiologist wastes valuable time during the work shift attempting to assess task difficulty from incomplete information.

In general, the radiologist does not have sufficient information in order to accurately prospectively assess the difficulty of a given radiology reading task. The screen size limits the number of features that can be displayed. Many features important for assessing complexity are not displayed in the summary-style work list as they would require more text. Even if the radiologist has access to all relevant features as well as available patient history (which is not always the case from the radiology workstation, e.g. it may not be connected with the EHR or EMR), it would be complex and time consuming to determine the best selection.

The work list provides limited patient information for each reading task, such as Patient identification, imaging modality, date and time of the radiology examination. The radiologist may employ various rules-of-thumb in assessing task difficulty from this limited information, such as assuming that reading tasks for very old or very young patients are likely to be more difficult, but this provides only an approximate, and often inaccurate, prospective difficulty assessment. Without the age, radiologists can create some mental guesses that babies for example have higher PatientIDs, whereas people with much lower PatientIDs have been in the system much longer and are likely to be older. This can be wrong however, since patients may be new to the area, regardless of age. RVU value of an examination, is determinable but not shown. It is commonly driven by the modality and type of exam (e.g. by medical procedure code), which radiologists generally understand. However, as already discussed, RVU value is usually not sufficient information to accurately assess the difficulty of the reading.

The radiologist could make a better prospective difficulty assessment for a given reading task by selecting that task from the work list and reviewing the additional information beyond the radiology reading display, but this would take as long as the reading itself in some cases. This information may include, for example, the number of images in the examination, the reason for the examination, data from past radiology reports, and so forth. However, this information is still limited, because the PACS generally does not provide access to the general-purpose EMR or EHR. Moreover, even if sufficient information for prospective difficulty assessment is made available by selecting the work item, this approach has several disadvantages. First, the radiologist must expend valuable time in selecting the work item and comprehending the additional information provided for the selected work item. As previously noted, a radiologist may be expected to perform a complete reading from selecting the item from the work list to dictating and filing the final radiology report in as little as 1.5-2.0 minutes in this setting, even taking 30 seconds to select a work item and comprehend the additional patient information provided and thereby prospectively assess reading difficulty is problematic. Furthermore, in a multi-radiologist work shift setting, selecting a task from the work list simply to review the patient data, without actually performing the reading task, may cause problems since other radiologists will assume that the selected reading task is being handled.

It is thus recognized herein that a problem with existing radiology workstations is that (1) the quantity and quality of patient information provided by the radiology workstation is limited, especially at the work list level; (2) this information deficit limits the ability of a radiologist to accurately prospectively assess reading task difficulty; and (3) in the intense radiology reading environment, this inability to accurately prospectively assess reading task difficulty produces disadvantages such as increased radiologist stress, reduced radiology reading throughput (measurable as reduced RVU points per shift), and potentially decreased radiology reading accuracy.

In view of these recognized problems, an improved radiology workstation is disclosed herein in which the work list is augmented by adding challenge level indicators (e.g. annotations or icons) to reading tasks of the work list. The challenge level provides a prospective assessment of the difficulty of the reading task, thus enabling the radiologist to make a more appropriate selection of the next reading task to perform. In some embodiments, the challenge level of a given radiology reading task is assessed on the basis of information contained in the PACS, without resort to an EMR, EHR, or other additional database. Patient data available on the PACS but not shown on the work list, such as the modality of the radiology examination to be read, and/or the reason for the radiology examination, and/or patient demographic data (both that shown on the work list and optionally additional patient demographic data that may be available on the PACS but not included in the work list) may be leveraged as relevant data for the prospective challenge level assessment. In some embodiments, past radiology examination information stored in the PACS is leveraged to provide a more accurate prospective assessment of the challenge level. For example, the number of previously acquired radiology images (prior to the current examination to be read), and/or the number of past radiology examinations, in a specific time window (e.g. the last week) may be considered as relevant data for the challenge level assessment. Additionally or alternatively, any past radiology reports for the patient, which are typically available on the PACS, may be mined to obtain relevant data for the challenge level assessment. Metadata associated with the images of the radiology examination to be read, such as the number of images, image resolution, or so forth, may additionally or alternatively be leveraged.

In some suitable embodiments, the challenge level assessment is computed as a weighted combination of various such factors. The weights assigned to the various factors may be chosen globally, or in some embodiments at least some weights may be chosen as radiologist-specific weights, for example reflecting the impact of individualized radiologist expertise on the challenge level assessment made for a particular radiologist. In some embodiments, these weights (global and/or radiologist-specific) may be adaptive weights that are adjusted based on feedback in the form of actual radiology examination reading times.

The challenge level may represent an absolute assessment of difficulty of a reading task. However, since the goal is generally to maximize the number of RVU points per work shift, in some embodiments the challenge level for a task is adjusted based on the RVU points for that task. Intuitively, Challenge value=RVU points/(difficulty or reading time) provides a rational basis by which the radiologist can maximize RVU points per shift. This formulation computes the challenge level in terms of RVU per unit time ($/hour). In this formulation, a high-challenge (or “difficult”) task provides low RVU per unit time; whereas, a low-challenge (or “easy”) task provides high RVU per unit time. Alternatively, the inverse can be used (Challenge value=estimated reading time/RVU point) in which case a high-challenge task has a long reading time/RVU point.

It will be appreciated that the disclosed approaches have numerous advantages. They improve the performance of the radiology workstation by providing the radiologist with prospective challenge level indicators annotated to reading tasks of the work list. These challenge level indicators provide the radiologist with this information in a form that can be immediately grasped, and encapsulate information from diverse sources to provide a prospective assessment of reading difficulty that solves the problem recognized herein that the radiologist (in the absence of such indicators) has insufficient information to effectively select the next radiology examination for reading.

As used herein, a “patient” refers to a radiology examination subject (or “examination subject” for brevity). The term “patient” as used herein broadly encompasses hospital in-patients, hospital out-patients, emergency room patients, independent imaging center clients, persons who visit a medical office of any kind and are directed to a radiology laboratory for a radiology examination, or so forth.

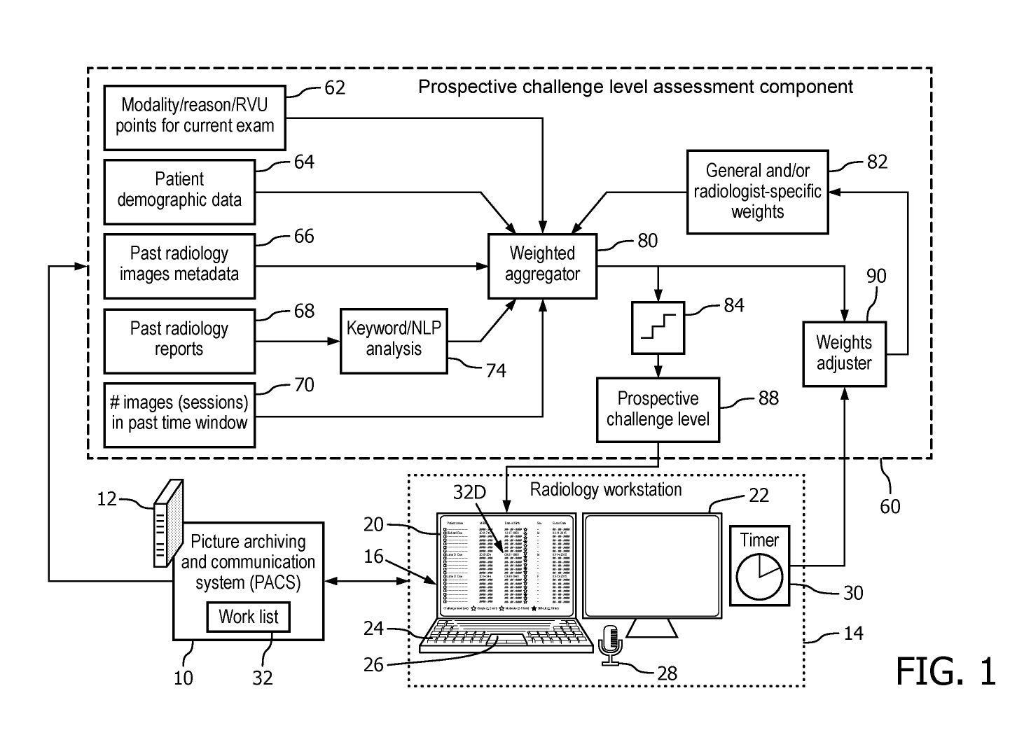

With reference to FIG. 1, a Picture Archiving and Communication System (PACS) 10 is implemented on a networked computing system 12 diagrammatically indicated in FIG. 1 by a server computer. It will be appreciated that the networked computing system 12 may comprise a single server computer, a computing cluster, a cloud computing resource, or so forth. The PACS 10 installed on the networked computing system 12 is connected with one or (more typically) a plurality of radiology workstations, where FIG. 1 illustrates a single representative radiology workstation 14, via a secure electronic data network, such as a wired and/or wireless Wide Area Network (WAN) implemented via Ethernet, WiFi, or another suitable wired and/or wireless electronic data networking protocol. The secure electronic data network should have sufficient bandwidth to communicate radiology images, which are typically large data files, to and from the radiology workstation 14. Optionally, the PACS 10 installed on the networked computing system 12 may be connected with other computing systems such as physician's desktop computers, radiological imaging system controllers (e.g. MRI or CT system controllers) or so forth (not shown).

Each radiology workstation 14 includes a workstation electronic processor, for example embodied as a computer 16. The workstation electronic processor may be a multi-core processor, a cloud computing resource, or so forth. Each radiology workstation 14 further includes at least one display device, e.g. an illustrative display device 20 of the computer 16 and an additional display device 22. It is contemplated that the radiology workstation 14 may employ a web browser-based user interface. Providing the radiology workstation with two (or more) display devices can be advantageous as it allows one display device to be used to display textual content or other auxiliary information while the other display device is used as a dedicated radiology image viewer; however a radiology workstation with only a single display device is also contemplated. At least one display device of the radiology workstation should be a high-resolution display capable of displaying radiology images with sufficiently high resolution to enable the radiologist to accurately read the radiology image. Each radiology workstation 14 further includes at least one user input device, such as: an illustrative computer keyboard 24; a mouse, touchpad 26, or other pointing device; a touch-sensitive display (e.g., one or both display devices 20, 22 may be a touch-screen display); a dictation microphone 28, or so forth. Optionally, the radiology workstation 14 is further capable of measuring a reading time defined between selection of a radiology examination reading task and completing receipt of the entry of the radiology report for that task. This reading time measurement is diagrammatically indicated in FIG. 1 by a diagrammatic timer 30; however, it will be appreciated that the timer 30 is more typically implemented by the computer 16, e.g. using the internal (i.e. system) clock of the computer 16.

The term “Picture Archiving and Communication System” or “PACS” as used herein broadly encompasses any electronic database that stores radiology images acquired during radiology examinations and provides retrieval access for the stored radiology images. The PACS is distinct from general-purpose medical databases such as the Electronic Medical Record (EMR) or Electronic Health Record (EHR), although some integration of the PACS with a general-purpose medical database is contemplated. For example, the patient record in the EMR or EHR may include hyperlinks to radiology examinations stored in the PACS, and/or the PACS record for a patient may include a hyperlink to the patient's record in the EMR or EHR. In typical embodiments, the PACS stores radiology images in accordance with the Digital Imaging and Communications in Medicine (DICOM) file format definition promulgated by the National Electrical Manufacturers Association (NEMA), or in a variant of the standard DICOM definition.

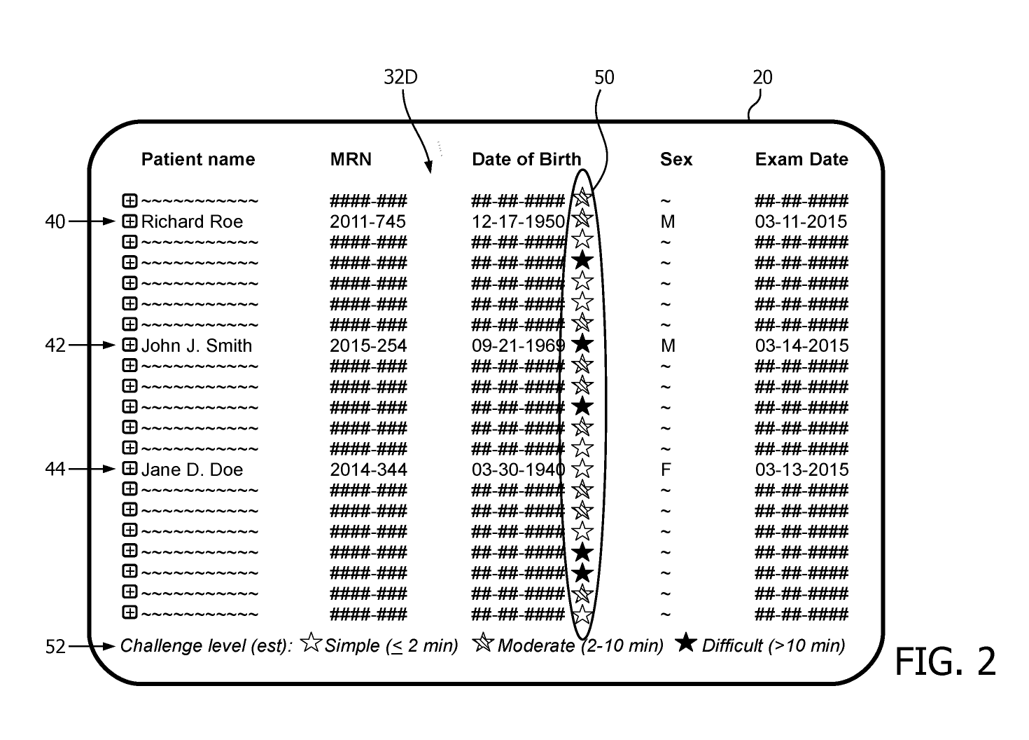

With continuing reference to FIG. 1 and with further reference to FIG. 2, the radiology workstation 14 operating in conjunction with the PACS 10 installed on the networked computing system 12, provides a work environment for a radiologist as follows. A work list 32 is maintained which includes radiology examination reading tasks that have not yet been performed (i.e. for which a radiology report has not yet been entered or stored in the PACS 10). The illustrative work list 32 is maintained on the PACS 10 which is convenient in the case of a larger radiology department that may have two or more radiologists working a single shift via two or more instances of the illustrative radiology workstation 14 in this arrangement the same work list 32 is then accessed by each radiologist so that they can mutually track remaining reading tasks. Alternatively, it is contemplated for the work list to be maintained at the radiology workstation, which may be appropriate in a setting in which only a single radiology workstation is provided. The work list 32 is displayed as a work list display 32D on a display device of the radiology workstation in illustrative FIGS. 1 and 2, the work list display 32D is displayed on the computer display device 20, although in other embodiments it might be displayed on the second display device 22, or the radiology workstation 14 may optionally be configured to display the work list display 32D on a selectable one of the display devices 20, 22. The illustrative display 32D of the work list 32 shows, for each radiology examination reading task, a number of data fields identified by respective headings: “Patient name”, “MRN” (where “MRN” stands for “Medical Record Number”, or equivalently, PatientID), “Accession Number”, “Date of Birth”, “Sex”, and “Exam(ination) Date”. Accession number refers to the current image(s), typically of the same modality taken at the same imaging event. These are merely illustrative data fields, and additional or other data fields are contemplated to be displayed in the display of the work list 32. For illustrative purposes, the display 32D of the work list 32 includes three illustrative radiology examination reading tasks: a reading task 40 for patient “Richard Roe”; a reading task 42 for patient “John J. Smith”; and a reading task 44 for patient “Jane D. Doe”. The remaining illustrative radiology examination reading tasks of the display 32D are diagrammatically indicated using placeholder symbols “˜” (tilde) and “#” (pound sign).

A radiologist viewing the work list display 32D on the display device 20 of the radiology workstation 14 chooses a next radiology examination reading task from the work list 32. Upon receiving this selection via at least one user input device 24, 26, 28, the radiology workstation 14 retrieves one or more radiology images of the selected radiology examination reading task from the PACS 10 and displays the retrieved radiology images, e.g. on the display device 22. This display may incorporate usual image display or rendering techniques such as zoom, pan, resizing, displaying selected images side-by-side or in another arrangement, allowing the radiologist to use on-screen cursors to perform spatial and/or intensity measurements, or so forth. It will be appreciated that only one image, or a subset of a set of images, or all images, may be displayed at any given time during the reading process. For example, the radiologist may choose to work through a set of image slices one-by-one so that only a single image slice is displayed at any given time. Optionally, the radiologist may bring up and display images from other radiology examinations, e.g. to compare a current tumor image with one acquired in an earlier radiology examination to observe growth or shrinkage of the tumor. During the reading, the radiology workstation 14 receives, via the at least one user input device, entry of a radiology report for the selected radiology examination reading task. In a common approach, the dictation microphone 28 is used to receive entry of an orally dictated radiology report; however, it is additionally or alternatively contemplated to employ another user input device, such as using the keyboard 24 to type in the radiology report or to edit the initially orally dictated report. When the radiologist is satisfied with the entered radiology report for the selected radiology examination reading task, the radiologist performs suitable operations to save the report in the PACS 10, send the report to the patient's physician, or otherwise store and/or disseminate the report. For example, the radiology workstation 14 may display a “file report” button or the like which can be selected by the radiologist using a pointer or the like to execute the filing of the report. The selected radiology examination reading task is converted to a completed radiology examination reading task upon filing of the entered radiology report (i.e. storing the radiology report on the PACS 10), and the work list 32 is updated by removing the completed radiology examination reading task from the work list and displaying the updated work list on the radiology workstation 14. The radiologist will then move on to view the work list display 32D on the display device 20 (which may be automatically brought up in response to filing the radiology report for the last examination, and/or may be brought up by a suitable activation operation performed by the radiologist such as clicking on the entry) The radiologist then performs the next radiology examination reading task as just described.

In selecting a next radiology examination reading task from the work list 32, the radiologist is conventionally limited to the few items of information displayed for each reading task on the work list display 32D. In illustrative FIG. 2, these include “Patient name”, “MRN” “Date of Birth”, “Sex”, and “Exam Date”. From this limited information, it is difficult for the radiologist to decide which reading task to perform next. The radiologist may, for example, want to perform a simple task if the last task was difficult (or vice versa), but the conventionally displayed information only enables the radiologist to make an educated guess at which examinations are simple (or difficult), for example based on patient age (discerned from “Date of Birth” and assuming very young or very old patients will have more difficult readings), or based on the exam date (if an examination has an “old” date it may be surmised it has been passed over previously as a difficult reading).

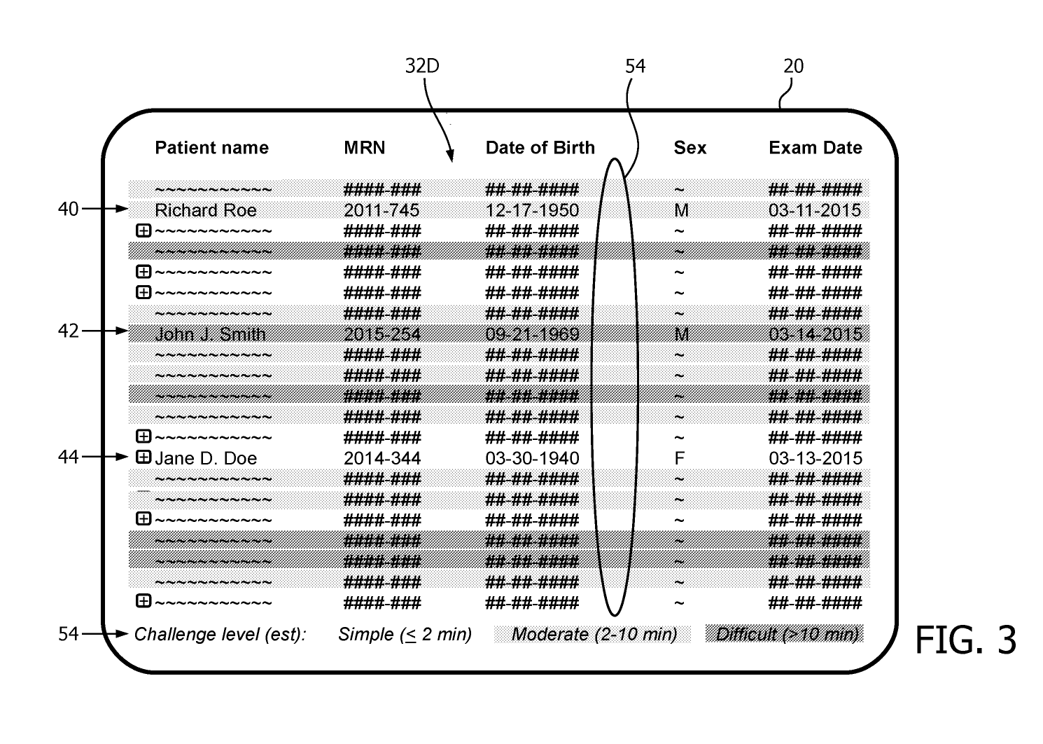

With continuing reference to FIG. 2, to aid the radiologist in assessing examination reading difficulty, each radiology examination reading task listed on the work list display 32D includes an indicator of the prospective challenge level. In illustrative FIG. 2, these indicators are presented as different icons 50, whose meanings are indicated in a challenge level legend 52 at the bottom of the display. The illustrative iconography employs: an open star to indicate a simple reading (estimated reading time of 2 min or less); a grayish star to indicate a moderately challenging reading (estimated reading time of 2-10 min); and a filled (black) star to indicate a difficult, i.e. challenging, reading (estimated reading time of greater than 10 min). While shading levels are used in FIG. 2, the stars can alternatively be color-coded, e.g. a green star for a simple examination, a yellow star for a moderately challenging examination, and a red star for a difficult examination. Additionally or alternatively, different icons can be used to indicate difficulty, e.g. a one-star icon for easy, a two-star icon for moderate, and a three-star icon for difficult. Shapes other than stars may also be used as the icons, e.g. circular icons rather than stars, or different shapes for different challenge levels. The number of challenge levels can also be more than three, although since the goal is to aid the radiologist in rapidly assessing the difficulty of different reading tasks the number of challenge levels should be relatively low, e.g. preferably no more than four or five levels, though six or more levels are also contemplated. With brief reference to FIG. 3, in other embodiments the indicator takes the form of a color-coded background 54 for the text of the radiology examination reading task, with a challenge level legend 56 at the bottom of the display indicating the meaning of the different backgrounds. In FIG. 3, a white background indicates a simple task, a light gray background indicates a moderate task, and a dark gray background indicates a difficult task. Alternatively, the backgrounds 54 may be of different colors, e.g. white for simple, yellow for moderate, and red for difficult. The color indicator may alternatively be different colors for the text rather than the background. In another approach (not shown), the indicators may take the form of unadorned text, e.g. an additional column in the work list display listing the challenge level for each task as “simple”, “moderate”, or “difficult”. Such textual indicators could also be numeric, e.g. listing the challenge level as an estimated reading time in minutes for each task.

It is also contemplated for the radiology workstation 14 to include user inputs or configuration settings that allow the radiologist to configure the indicator format, as some radiologists may (by way of illustration) prefer color-coded backgrounds 54 (FIG. 3) as being very easy to visually detect, whereas other radiologists may prefer separate color-coded icons (FIG. 2) because the colorized backgrounds may obscure the textual content.

Moreover, while the legends 52, 56 are convenient for indicator interpretation, they occupy valuable screen space and may optionally be omitted (either always or as a user-selectable “show/hide” option).

It will be appreciated that the challenge level indicators of FIGS. 2 and 3 are prospective challenge level indicators, as they are computed for not-yet-read radiology examination tasks, i.e. before the radiologist has entered the radiology report for the task. As a consequence, the determination of the challenge level indicators cannot rely upon content of the radiology report for the (current) radiology examination reading task, although as disclosed herein the determination may optionally rely in part on radiology reports for past radiology examinations of the examination subject (i.e. patient) of the radiology examination reading task.

Although not illustrated, it is contemplated to order the reading tasks of the work list by challenge level, e.g. listing the highest challenge level tasks at the top (or vice versa). For example, the work list may include an ordering selection input via which the user can choose to order the tasks of the work list by any column (e.g. by Patient Name, alphabetically; by MRN ascending or descending; by date-of-birth ascending or descending; et cetera, and ordering by challenge level is one of the ordering options presented to the radiologist.

With reference back to FIG. 1, the challenge level indicators are generated by a prospective challenge level assessment component 60 comprising an electronic processor programmed to generate a prospective challenge level for a radiology examination reading task prior to the radiology workstation receiving entry of the radiology report for the radiology examination reading task. The prospective challenge level assessment component 60 may be implemented on (i.e. comprise) the workstation electronic processor (e.g. computer 16) of the radiology workstation 14. Alternatively, the prospective challenge level assessment component 60 may be implemented on (i.e. comprise) the electronic processor (e.g. server 12) of the PACS 10. In some embodiments, the prospective challenge level assessment component 60 generates the prospective challenge levels for the radiology examination reading tasks using only data stored on the PACS 10. This advantageously allows the prospective challenge level assessment component 60 to be self-contained on the radiology workstation computer 16 and/or on the server 12 of the PACS, being operational with access only to the PACS 10. It also allows the prospective challenge level assessment component 60 to be computationally efficient since it does not need to access any other databases. However, it is also contemplated for the prospective challenge level assessment component 60 to generate the prospective challenge levels for the radiology examination reading tasks using data stored on the PACS 10, which may include historical images and reports, as well as data from another source such as an Electronic Medical (or Health) Record (EMR or EHR).

In the illustrative embodiment the prospective challenge level assessment component 60 used only data available on the PACS 10. This information includes current examination information 62 stored on the PACS for the current radiology examination, such as the reason for examination, the imaging modality of the examination, and/or the number of RVU points for the examination. The reason for examination is typically indicated by the ordering physician, and is commonly (though not necessarily) stored as an International Classification of Diseases (ICD-9) code which is a standard classification system used by medical institutions, medical insurance companies, and the like. The imaging modality may be obtained from the examination metadata or from metadata of individual images. For example, the standard DICOM header includes a field for specifying the image modality. The RVU points are generally a function of imaging modality and possibly ICD-9 code, and hence can be calculated. For example, in one common counting scheme, an MRI is assigned 8 RVU points, a CT is assigned 4 RVU points, and an x-ray is assigned 1 RVU point (of course, a different counting scheme may be employed at a given medical institution). Other metadata of the current radiology examination and/or the DICOM headers of the images may also be used in quantifying the challenge level of the radiology examination, such as the examination date, the number of images in the examination, image size/resolution, or so forth.

Some basic patient demographic information 64 is also available on the PACS 10. This is the demographic information for the examination subject of the selected radiology examination reading task. As already mentioned, such data generally include at least sex and date of birth, and may also include data such as ethnicity.

Further relevant information available on the PACS 10 may include information past radiology examinations, such as metadata 66 associated with past examinations/images, past radiology reports 68, and a count 70 of the number of past examinations (and/or the number of images of those past examinations) in one or more past time windows (e.g. in the last week, or in the last month, et cetera). The metadata 66 of past examinations/images that can be extracted is similar to that already described for the current examination data 62, e.g. ICD-9 exam code, imaging modality, or so forth.

In the case of past radiology reports 68, these generally include detailed information such as findings of interest such as tumor, anatomical abnormalities, or indications of disease as well as measurements and summary impressions such as recommendations for future imaging, surgery, or blood tests. Indications of surgery can increase the reading difficulty due to scar tissue, for example. There is also available meta-data for the report such as patient name, gender, patient MRN, date of birth, completion time, and referring physician. As already mentioned, some or all of this information may already be available elsewhere on the PACS 10.

Additionally, however, the radiology report contains the clinical findings of the radiologist who prepared the past examination report, and may contain other information such as notes that certain anatomy appears normal (or abnormal, with a description). The findings may be broken down by type of anatomy (e.g. heart, lung, liver). The radiology report may also include the radiologist's impressions, i.e. the summary section where a diagnosis may be indicated. Key measurements of previously described key findings are also repeated. The radiology report (or at least the main body containing the summary section and radiologist notes) is typically written in freeform rather than as entries of structured data entry fields. Accordingly, the radiology report, or at least the freeform portion(s) thereof, are optionally analyzed by keyword searching and/or natural language processing (NLP) analysis 74 to extract relevant information. Using natural language processing (NLP) some key factors can be extracted from past reports which can add indications that may affect the level of difficulty such as past history of human immunodeficiency virus (HIV), cancer, trauma, surgery or recent indications (for example, within the last month) fever, cough, pain, headache, shortness of breath (SOB), or ‘sudden onset’.

The count or number 70 of past examinations and/or images in the designated time window (e.g. the last week) are readily obtained from the PACS 10 which stores the radiology examination data including the images. As optionally used herein for assessing the challenge level, if the examination subject of the selected (not-yet-read) radiology examination has had a large number of recent prior images/examinations, this tends to suggest the reading of the selected current (not-yet-read) radiology examination will be more challenging. This inference may be drawn by various rationales. In one rationale, the fact that the examination subject has had a number of radiology examinations in the recent past is suggestive that the examination subject has a significant medical condition, or perhaps a number of different medical conditions, which may complicate the reading. Under another rationale, the existence of a large number of radiology examinations in the recent past constitutes additional patient data that the radiologist may need to consult in performing the latest reading, again complicating the reading. (For example, the radiologist may need to refer back to several past examinations to assess the change over time of a cancerous tumor in response to oncology therapy). Under yet another rationale, a large number of examinations in the recent past may suggest that the patient's condition is difficult to diagnose (so that repeated and/or variant examinations have been ordered), yet again suggesting that the current reading will be more challenging.

The recent number of images in the past time window 70 is available only if the examination subject has in fact had one or more past radiology examinations in the designated time window. If this is not the case, then the count/number information has the value zero. While having a large number of recent past examinations can be suggestive of a more challenging reading as just described, having no prior examinations at all can make the reading more challenging for different reasons.

Counting all available images of the current body part easily accessible from storage, including those prior to the recent ‘Time Window’, it may be that there are no relevant images at all. With no relevant past examinations available, the radiologist cannot make comparisons with past findings, but instead must perform the current reading task ab initio, with no prior findings for guidance. Further, since anatomy varies, having a baseline from past examinations can indicate normal variations to compare with suspect parts of an image; without this baseline, the reading becomes more challenging.

Various components of these various data 62, 64, 66, 68, 70, and/or other data, may be available on the PACS 10 for assessing the challenge level of any particular selected radiology examination reading task. The prospective challenge level assessment component 60 may be configured to collect and use any portion, or all, of the available data.

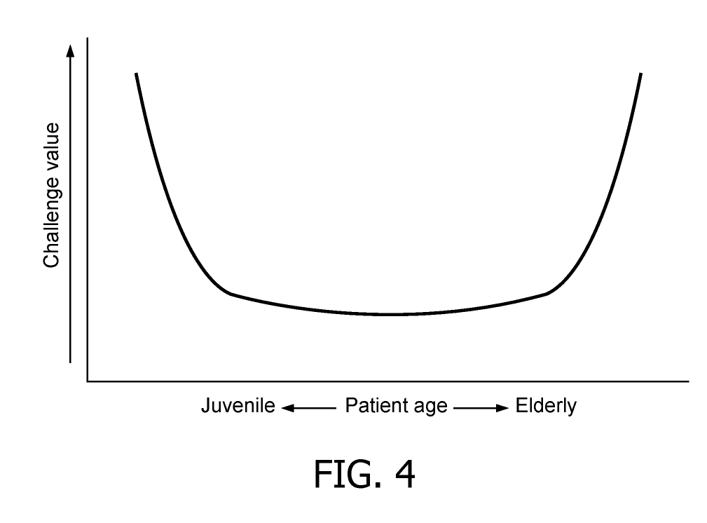

With reference to FIG. 4, for example, a simple approach for quantitatively assessing challenge level is to identify patient age (e.g. from date of birth) and map the patient age to a challenge level, for example using the generally “U”-shaped transform curve of FIG. 4 which indicates an increasing prospective challenge for juvenile and elderly patients and lowest prospective challenge for adult (but not elderly) patients. The resulting challenge value is continuous, and may be thresholded to generate a challenge level (e.g. one of three discrete values, simple, moderate, or difficult, as in the examples of FIGS. 2 and 3).

Data 68 from past radiology reports may be used to estimate the prospective challenge value in various ways. For example, it is straightforward to identify various diseases based on occurrence of the disease name in the findings section of the radiology report. In most cases, if a disease name is mentioned it may be reasonable to assume that the examination subject either has that disease or at least is suspected to have that disease. Optionally, NLP may be employed to distinguish between, for example, a statement that the image indicates the disease versus a statement that the image does not indicate the disease. Since certain diseases make certain diagnostic tasks more difficult, the association of a particular disease with a patient (possibly in combination with the examination task as indicated by the current examination ICD-9 code) can be used to increase or decrease the assessed challenge value.

The imaging modality of the current examination can be further used in assessing the challenge value. For example, the average reading time varies for different modalities due to differing numbers of images (e.g. a typical x-ray examination has only a few images while an MRI or CT examination may have hundreds of slices or images) and the complexity of the image content (again, an MRI image is usually more complex than an x-ray image). For example, the following are typical reading times for various imaging modalities: 1.5 min for an x-ray; 2 min for an ultrasound; 5 min for a CT; and 7 min for an MRI.

The RVU points are optionally taken into account in assessing challenge level. For discussion, RVU=f(medical procedure code) is used, where again the procedure code may be, for example, a CPT or HCPCS code based on modality and body part generally. In general, the goal when assigning RVU points is to assign more RVU points to more challenging reading tasks. Thus, if RVU points assignment for a reading task is not taken into account then it will generally be the case that reading tasks with low RVU points will be assigned a lower challenge level than those with higher RVU points. (This is not always the case, because as already discussed the RVU assignment methodology may take into account various factors, some of which may be unrelated to reading difficulty). If the goal of the challenge level indicators is to indicate the absolute difficulty, then this is appropriate. On the other hand, if the goal is to assist the radiologist in maximizing the accumulation of RVU points over the course of a work shift, then the challenge level should take into account RVU points, or in other words the challenge level should be computed for a given RVU point value. One way to do this is to divide the difficulty assessment by the RVU points in order to generate the challenge level in terms of time/RVU point. Lower time/RVU values equate to an ‘easy or more desirable’ selection for the radiologist.

With continuing reference to FIG. 1, more generally the prospective challenge level assessment component 60 may compute the challenge value as a weighted aggregation 80 of various data values, with challenge value components computed from the data being weighted by respective weights 82, which may be general weights (the same weight being used for computing the challenge value for all radiologists) or radiologist-specific weights (different weights used to account for different skills/preferences of different radiologists). The aggregation 80 may be a weighted sum, or additional manipulation may be applied (e.g. normalizing or otherwise scaling challenge level components). For maximum flexibility, some or all challenge values components contributing to the aggregation 80 may be continuous values, so that the output of the aggregation 80 is a continuous-valued challenge value. A quantization operation 84 (e.g. a multi-level thresholder) then converts the continuous-valued challenge value to a discrete challenge level 88 that is used to generate the indicators (e.g. indicator stars 50 of FIG. 2, or indicator colors in accord with the legend 56 as illustrated in FIG. 3).

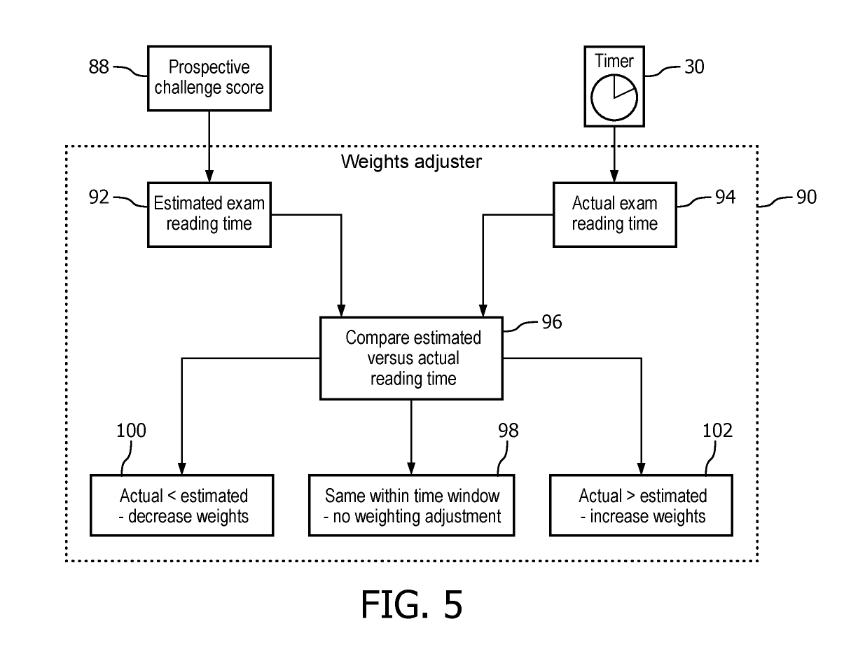

With continuing reference to FIG. 1 and with further reference to FIG. 5, in some embodiments the weights 82 are adaptive weights that is, a weights adjuster 90 adjusts the weights 82 (or at least one or more of the weights 82) automatically in order to align the reading time estimated by the challenge level 88 with actual reading time measured by the timer 30. FIG. 5 diagrammatically illustrates a suitable embodiment of the weights adjuster 90. An estimated examination reading time 92 is determined from the prospective challenge level 88. In the examples of FIGS. 2 and 3 this is straightforward: the “simple” challenge level translates to an estimated reading time of 2 min or less; the “moderate” challenge level translates to an estimated reading time of 2-10 min; and the “difficult” challenge level translates to an estimated reading time of greater than 10 min. (Typically the estimated reading time 92 is a time window, though a single estimated average reading time is contemplated). On the other hand, the radiologist selects a radiology examination reading task to perform the timer 30 starts and measures the time from this selection until the radiologist files the entered radiology report this time interval is the actual reading time 94. A comparison 96 is made between the estimated reading time 92 and the actual reading time 94. If this comparison indicates that the actual reading time 94 is within the time window of the estimated reading time 92 (e.g. is 2 min or less for in the “simple” case, or 2-10 min in the “moderate” case, or greater than 10 min in the “difficult” case), then the weights 82 are appropriate and no adjustment 98 is made. If, however, the actual reading time 94 is less than the estimated examination reading time window 92 then the weights are decreased 100 so that the challenge level estimate is lowered. Conversely, if the actual reading time 94 is greater than the estimated examination reading time window 92 then the weights are increased 102 so that the challenge level estimate is raised. The amount of lowering or raising 100, 102 is suitably chosen based on how far the actual reading time 94 is outside the estimated time window 92.

It will be appreciated that the illustrative computational components such as the prospective challenge level assessment component 60 may be embodied as a non-transitory storage medium storing instructions executable by an electronic processor (e.g. the workstation computer 16, or the PACS server 12) to perform the disclosed computations. The non-transitory storage medium may, for example, comprise a hard disk drive, RAID, or other magnetic storage medium; a solid state drive, flash drive, electronically erasable read-only memory (EEROM) or other electronic memory; an optical disk or other optical storage; various combinations thereof; or so forth.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A radiology reading device comprising:
a radiology workstation including a workstation electronic processor, at least one user input device, and at least one display device, the radiology workstation configured to:
display a work list of radiology examination reading tasks,
receive via the at least one user input device a selection of a radiology examination reading task from the work list, and
retrieve one or more radiology images of the selected radiology examination reading task from a Picture Archiving and Communication System (PACS) and display the retrieved radiology images; and
a challenge level assessment component comprising an electronic processor programmed to generate prospective challenge levels for the radiology examination reading tasks of the work list;
wherein the radiology workstation is configured to display the work list of radiology examination reading tasks with indicators of the prospective challenge levels generated by the challenge level assessment component for the radiology examination reading tasks.

2. The radiology reading device of claim 1 wherein the challenge level assessment component comprises the electronic processor programmed to generate a prospective challenge level for a radiology examination reading task from data including at least radiology images acquired in past radiology examinations of the examination subject of the radiology examination reading task.

3. The radiology reading device of claim 1 wherein the challenge level assessment component comprises the electronic processor programmed to generate a prospective challenge level for a radiology examination reading task from data including at least metadata associated with radiology images acquired in past radiology examinations of the examination subject of the radiology examination reading task.

4. The radiology reading device of claim 1 the challenge level assessment component comprises the electronic processor programmed to generate a prospective challenge level for a radiology examination reading task from data including at least a count of radiology images acquired in past radiology examinations of the examination subject of the radiology examination reading task over a predefined past time window.

5. The radiology reading device of claim 1 wherein the challenge level assessment component comprises the electronic processor programmed to generate a prospective challenge level for a radiology examination reading task from data including at least past radiology reports for past radiology examinations of the examination subject of the radiology examination reading task.

6. The radiology reading device of claim 5 wherein the challenge level assessment component comprises the electronic processor programmed to generate a prospective challenge level for a radiology examination reading task from keywords extracted from past radiology reports for past radiology examinations of the examination subject of the radiology examination reading task.

7. The radiology reading device of claim 5 wherein the challenge level assessment component comprises the electronic processor programmed to generate a prospective challenge level for a radiology examination reading task from data extracted from Natural Language Processing (NLP) of past radiology reports for past radiology examinations of the examination subject of the radiology examination reading task.

8. The radiology reading device of claim 1 wherein the challenge level assessment component comprises the electronic processor programmed to generate a prospective challenge level for a radiology examination reading task using only data stored on the PACS.

9. The radiology reading device of claim 1 wherein the challenge level assessment component comprises the electronic processor programmed to generate a prospective challenge level for a radiology examination reading task by operations including:
extracting data on one or both of the radiology examination reading task and the examination subject of the radiology examination reading task;
computing a weighted quantitative aggregation of the extracted data; and
thresholding or quantizing the weighted quantitative aggregation to generate the prospective challenge level for the radiology examination reading task.

10. The radiology reading device of claim 9 wherein the challenge level assessment component comprises the electronic processor further programmed to update weights of the weighted quantitative aggregation based on comparison of an estimated reading time generated from the computed weighted quantitative aggregation and an actual reading time between the radiology workstation receiving the selection of the radiology examination reading task from the work list and completing entry of a radiology report for the selected radiology examination reading task.

11. The radiology reading device of claim 1 wherein the indicators of the prospective challenge levels generated by the challenge level assessment component for the radiology examination reading tasks are color indicators, icon indicators, colored icon indicators, or textual indicators.

12. (canceled)

13. (canceled)

14. A radiology reading method comprising:
generating, using an electronic processor, a prospective challenge level for each radiology examination reading task of a work list of radiology examination reading tasks that have not yet been performed; and
displaying the work list on a radiology workstation with each radiology examination reading task of the displayed work list annotated by an indicator of the prospective challenge level generated for the radiology examination reading task.

15. The radiology reading method of claim 14 further comprising:
receiving from the displayed work list a selection of a radiology examination reading task via the radiology workstation;
retrieving from a Picture Archiving and Communications System (PACS) one or more radiology images of the selected radiology examination reading task;
displaying the retrieved one or more radiology images on the radiology workstation;
receiving via the radiology workstation a radiology report for the selected radiology examination reading task;
storing the radiology report for the selected radiology examination reading task on the PACS whereby the selected radiology examination reading task is converted to a completed radiology examination reading task; and
after the storing, updating the work list by removing the completed radiology examination reading task from the work list and displaying the updated work list on the radiology workstation.

16. The radiology reading method of claim 14 wherein the generating is performed using only information stored on a Picture Archiving and Communications System (PACS), wherein the information includes at least one of:
data mined from radiology is stored on a Picture Archiving and Communications System (PACS,
data mined from radiology reports stored on a Picture Archiving and Communications System (PACS); and
radiology examination subject demographic data stored on a Picture Archiving and Communications System (PACS).

17. (canceled)

18. (canceled)

19. (canceled)

20. The radiology reading method of claim 14 wherein the generating comprises, for each radiology examination reading task:
extracting data on one or both of the radiology examination reading task and the examination subject of the radiology examination reading task;
computing a weighted quantitative aggregation of the extracted data; and
thresholding or quantizing the weighted quantitative aggregation to generate the prospective challenge level for the radiology examination reading task.
